(19) United States
(12) Patent Application Publication
(10) Pub. No.: US 2021/0207209 A1
(43) Pub. Date: Jul. 8, 2021

(54) GENE SEQUENCING CHIP, GENE SEQUENCING METHOD, GENE SEQUENCING DEVICE

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Fengchun PANG, Beijing (CN); Peizhi CAI, Beijing (CN); Yue GENG, Beijing (CN)

(21) Appl. No.: 16/073,364

(22) PCT Filed: Jan. 10, 2018
(86) PCT No.: PCT/CN2018/072062

§ 371 Date: Jul. 27, 2018

(30) Foreign Application Priority Data
Apr. 21, 2017 (CN) 201710265434.8

(51) Int. Cl. C12Q 1/6869; C12Q 1/6837

(52) U.S. Cl. CPC C12Q 1/6869; C12Q 1/6837

(57) ABSTRACT

A gene sequencing chip, a gene sequencing method, and a gene sequencing device are disclosed. The gene sequencing chip includes a display panel which includes a plurality of display units, where each of the display units includes a transistor and an electrode connected with a first pole of the transistor; an opening defining layer which is arranged on the display panel and includes openings corresponding with the display units in a one-to-one manner; and an ion-sensitive film which is at least partially arranged in the openings and is connected with a control electrode of the transistor.

### RELATED APPLICATIONS

The present application is the U.S. national phase entry of PCT/CN2018/072062, with an international filing date of Jan. 10, 2018, which claims the benefit of Chinese Patent Application No. 201710265434.8, filed Apr. 21, 2017, the entire disclosures of which are incorporated herein by reference.

### TECHNICAL FIELD

The present disclosure relates to the field of gene sequencing technologies, and particularly to a gene sequencing chip, a gene sequencing method, and a gene sequencing device.

### BACKGROUND

A gene sequencing technique is among the most common techniques in the modern molecular biology study. The gene sequencing technique has been greatly developed from the first generation in 1977, and has successively went through Sanger sequencing technique invented by Frederick Sanger of the first generation, a high-throughput sequencing technique of the second generation, a single molecule sequencing technique of the third generation, and a nanopore sequencing technique of the fourth generation. The high-throughput sequencing technique of the second generation is currently still popular in the market.

The high-throughput sequencing technique of the second generation comprises the technique sequencing-by-synthesis invented by Illumina, the ionic semiconductor sequencing technique and the sequencing by litigation invented by Thermo Fisher, and the pyrophosphoric acid sequencing technique invented by Roche.

### SUMMARY

In some exemplary embodiments of the present disclosure gene sequencing chip is provided, having a display panel which includes a plurality of display units, where each of the display units includes a transistor and an electrode connected with a first pole of the transistor; an opening defining layer which is arranged on the display panel and includes openings corresponding with the display units in a one-to-one manner; and an ion-sensitive film which is at least partially arranged in the openings and is connected with a control electrode of the transistor.

For example, the display panel further includes a first substrate and a second substrate which are arranged oppositely; and a dielectric layer, a first fluid layer, and an electrically conductive second fluid layer which are arranged in a space between the first substrate and the second substrate.

For example, the first fluid layer is arranged on a side of the second fluid layer close to the electrode. The first fluid layer has a color different from the second fluid layer. The electrode and the second fluid layer are configured in such a manner that in case no electric field is formed, the first fluid layer is spread on a surface of the dielectric layer; and in case an electric field is formed, the first fluid layer is split into a plurality of subportions which are concentrated in regions of the dielectric layer where the corresponding transistors are located and which do not contact with one another.

For example, the transistor and the electrode are arranged on the first substrate.

For example, the gene sequencing chip further includes a protection layer which covers the transistor and the electrode, where the ion-sensitive film is connected with the control electrode through a via hole in the protection layer.

For example, the openings are micropores with an aperture of 1-100 μm.

For example, the dielectric layer is arranged on a side of the first fluid layer away from the second fluid layer.

For example, the dielectric layer is a hydrophobic layer, and the first fluid layer is an oil film.

For example, the hydrophobic layer is made from a liquid including a fluoropolymer. For example, the oil film is made from a liquid which includes at least one of hexadecane and silicone, and in which at least one of a pigment and a dye is dissolved.

For example, the first fluid layer has a black color.

For example, the ion-sensitive film is made from Si₃N₄.

For example, the gene sequencing chip further includes a peripheral circuitry, where a second pole of the transistor is electrically connected with the peripheral circuitry through a signal line.

In other exemplary embodiments of the present disclosure a gene sequencing device is provided, including the gene sequencing chip as described above; a processor which is configured to obtain the base sequence of DNA chains according to a display change produced on the display panel during gene sequencing.

For example, the gene sequencing device further includes an imaging circuit which is configured to record a pattern displayed at a bottom of the display panel away from the openings, where the processor is configured to obtain the base sequence of DNA chains according to the pattern.

In still other exemplary embodiments of the present disclosure a gene sequencing method is provided, using the gene sequencing chip as described above, and including the steps of adding DNA microbeads which include DNA chains into the openings for PCR amplification; adding a plurality of kinds of deoxy-ribo nucleoside triphosphates into the openings successively, where the DNA chains are complementarily paired with one of the plurality of kinds of deoxy-ribo nucleoside triphosphates to produce an electrical signal on the ion-sensitive film which turns on the transistor, so that a display change is produced on the display panel; and obtaining the base sequence of DNA chains according to the display change.

For example, adding the plurality of kinds of deoxy-ribo nucleoside triphosphates into the openings successively, where the DNA chains are complementarily paired with one of the plurality of kinds of deoxy-ribo nucleoside triphosphates to produce an electrical signal on the ion-sensitive film which turns on the transistor, so that the display change is produced on the display panel includes adding the plurality of kinds of deoxy-ribo nucleoside triphosphates into the openings successively, and applying a selected potential to the second fluid layer, so that under the action of the electric field between the second fluid layer and the electrode during complementary pairing in the openings, the first fluid layer is split into subportions which are concentrated in regions of the dielectric layer where the corresponding transistors are located and which do not contact with one another.

For example, the gene sequencing method further includes obtaining a pattern which is displayed at a bottom of the display panel away from the openings after the first fluid layer is split into the subportions which do not contact with one another.

For example, obtaining the base sequence of DNA chains according to the display change includes determining the base type on the DNA chains according to the specific type of the deoxy-ribo nucleoside triphosphates which are added when the pattern is produced.

For example, the plurality of kinds of deoxy-ribo nucleoside triphosphates are a plurality of kinds of reversibly terminating deoxy-ribo nucleoside triphosphates, and the gene sequencing method further includes removing the plurality of kinds of reversibly terminating deoxy-ribo nucleoside triphosphates which are added into the openings successively by washing, and adding a sulfhydryl reagent for detecting the base type at a subsequent position of the DNA chains.

### BRIEF DESCRIPTION OF THE DRAWINGS

In order to explain the technical solutions in the embodiments of the present disclosure more clearly, the drawings to be used in the description of the embodiments will be introduced briefly in the following, apparently, the drawings described below are only some embodiments of the present disclosure, the ordinary skilled person in the art, on the premise of not paying any creative work, can also obtain other drawings from these drawings.

FIG. 1 illustrates a structural view of a gene test chip in an embodiment of the present disclosure;

FIG. 2 illustrates a structural view of a gene test chip in an embodiment of the present disclosure; and

FIG. 3 illustrates a view of a display change during testing with the gene test chip shown in FIG. 2.

### DETAILED DESCRIPTION OF EMBODIMENTS

To make the objects, the technical solutions and the advantages of embodiments of the present disclosure more apparent, the technical solutions of the embodiments of the present disclosure will be described in detail hereinafter in conjunction with the drawings of the embodiments of the present disclosure. Apparently, the embodiments described hereinafter are only some embodiments of the present disclosure, but not all embodiments. Based the embodiments described hereinafter, other embodiments obtained by those skilled in the art should fall within the scope of the present disclosure.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

For example, the words “first”, “second” as well as similar words used in the patent application specification and claims of the present disclosure do not mean any sequence, quantity or importance, but are only used to distinguish different components. The term such as “comprises,” “comprising,” “comprises,” “comprising”, “contains” or the like means that an element or article prior to this term encompasses element(s) or article(s) listed behind this term and equivalents, but does not preclude the presence of other elements or articles. The terms indicating orientations or position relationships such as “one side”, “the other side” or the like which are based on the orientation or position relationship illustrated in the attaching drawings, and are only for facilitating and simplifying the description of the present disclosure, rather than meaning or implying that the mentioned apparatus or element must have a specific orientation or must be constructed or operate in a specific orientation, and therefore should not be understood as limitations to the present disclosure.

For example, in embodiments of the present disclosure, the deoxy-ribo nucleoside triphosphate can be selected according to the kind of the gene sequence to be sequenced. For example, as for common gene sequencing for humans and animals, the deoxy-ribo nucleoside triphosphate can include 5′-triphosphates, e.g., deoxyadenosine 5′-triphosphate (dATP), deoxyguanosine 5′-triphosphate (dGTP), deoxycytidine 5′-triphosphate (dCTP) and deoxythymidine 5′-triphosphate (dTTP). The corresponding base in these four 5′-triphosphates are A, G, C, and T. As understood by the person with ordinary skill in the art, other deoxy-ribo nucleoside triphosphates still exist in the field of sequencing. For example, the base is modified into 5-methylcytosine (m⁵C), 7-methylguanosine (m⁷G), or the like.

For example, in embodiments of the present disclosure, a transistor can be an electronic component with switching characteristic, e.g., a transistor capable of conducting an electrical signal. For example, the transistor can be a field-effect transistor (FET), a thin film transistor (TFT), or the like. According to the structural design of the transistor, the control electrode can be a gate, the first pole can be a source, and the second pole can be a drain. Alternatively, the control electrode can be the gate, the first pole can be the drain, and the second pole can be the source.

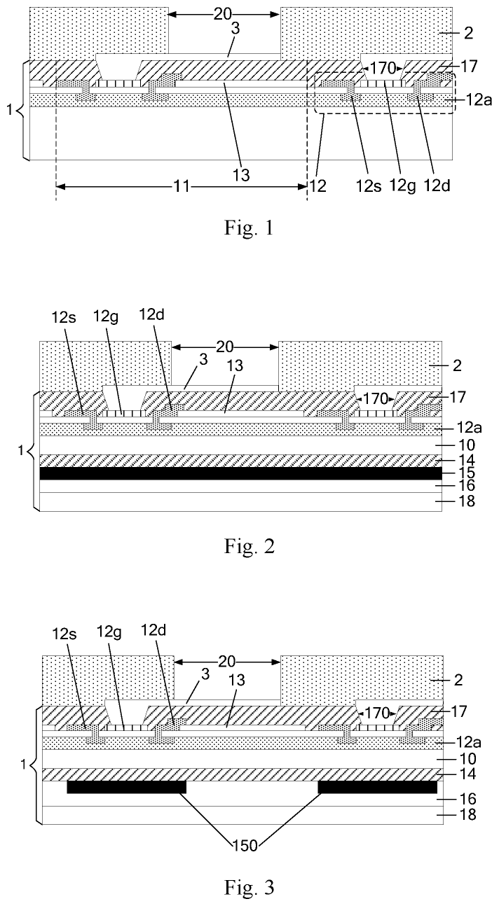

As shown in FIG. 1, embodiments of the present disclosure provide a gene sequencing chip including a display panel 1. The display panel 1 includes a plurality of display units 11. Each of the display units 11 includes a transistor 12 and an electrode 13. The electrode 13 is connected with a drain 12d of the transistor 12. The gene sequencing chip further includes an opening defining layer 2 on the display panel 1 which includes openings 20 corresponding with the display units 11 in a one-to-one manner; and an ion-sensitive film 3 which is at least partially arranged in the openings 20 and is connected with a gate 12g of the transistor 12.

For purpose of describing more clearly the gene sequencing chip of the present disclosure, the ionic semiconductor gene sequencing technique and the testing principle of the gene sequencing chip in embodiments of the present disclosure will be described hereinafter.

The ionic semiconductor gene sequencing method includes the step of pretreating a gene group DNA. Firstly, a DNA library is prepared. The gene group DNA is separated by a spraying method or the like. Namely, the DNA to be tested is cut into small fragments. Connector sequences are connected at both ends of each fragment, and each fragment is denatured into a single strand, so as to construct a single strand DNA library. These single strand DNA molecules are connected with microbeads (which are generally magnetic beads). Each microbead is connected with a single strand molecule. Then these microbeads are packaged in an emulsion into droplets of water in oil. Each droplet includes one microbead. Each fragment is amplified by PCR (Polymerase Chain Reaction) by a factor of about 1 million times, so as to form ten million template molecules to be measured. In this way, the amount of DNAs required for sequencing in the next step is obtained. The DNA microbeads including DNA chains are added into the openings 20 of the opening defining layer 2. During sequencing, the nucleotide molecules continuously flow one by one over the openings or micropores on the chip. In case a deoxy-ribo nucleoside triphosphate (dNTP) is complementarily paired with a DNA molecule in a specific micropore, the deoxy-ribo nucleoside triphosphate is synthesized into the DNA molecule, and a hydrogen ion (H⁺) is released. The hydrogen ion will induce a Nernst potential on a surface of the ion-sensitive film 3 (which can be made from Si₃N₄, etc). Since the ion-sensitive film 3 is connected with the gate 12g of the transistor 12. The potential signal is transmitted to the gate 12g, so as to turn on the transistor 12. A hydrogen ion is not released in a micropore where the DNA molecule is not complementarily paired, and no Nernst potential is induced on the surface of the ion-sensitive film 3. Accordingly, the transistor 12 corresponding with this micropore is not turned on. This causes a change in the display of the display panel 1. By means of a corresponding processor, the display change can be converted into corresponding digital electronic information, so as to obtain a base type in the DNA chains under test, and thus conduct gene sequencing.

Moreover, there are some notes for the structural composition of the gene test chip in embodiments of the present disclosure.

Firstly, the display panel 1 can include, but is not limited to, a liquid crystal display panel, an organic electroluminescence display panel, an electrowetting display panel, or the like, provided that the display panel 1 shows a change in display when the transistor 12 in different display units 11 is turned on or off. The change for example can be a change in display pattern.

Secondly, the transistor 12 can be a field-effect transistor (FET) which is prepared by a CMOS process, or a thin film transistor (TFT). Embodiments of the present disclosure are not limited in this regard. It is only required that the transistor 12 be an electronic component which has a switching characteristic and is capable of delivering corresponding electrical signals.

For example, the transistor 12 can be fabricated by a CMOS process. The transistor 12 is equivalent to a sensor sensitive to hydrogen ions. The transistor 12 includes a substrate (i.e., active layer) 12a which is a P type silicon substrate, and a source 12s and a drain 12d which are N type heavily doped silicon. The source 12s is connected with a peripheral circuitry (i.e., a processor chip) through a metallic signal line (which can be made from Al, Mo, or the like), and the drain 12d is connected with the electrode 13 (which can be made from ITO or the like).

The substrate of the transistor 12 in each of the display units 11 can be a one-piece component, or can be arranged individually. The source 12s of each of the transistors 12 can be connected to a same signal line to receive a same voltage signal.

In embodiments of the present disclosure, reference is made to the case in which the drain 12d of the transistor 12 is connected with the electrode 13. However, it will be appreciated by the person with ordinary skill in the art that, since the source and the drain of the transistor is interchangeable in term of structure and composition, it is also possible that the source 12s of the transistor 12 is connected with the electrode 13. Namely, the drain 12d of each of the transistors 12 is connected with a same signal line to receive a same voltage signal. This is an equivalent variant of the above embodiment.

Thirdly, since the gene sequencing chip includes the plurality of openings 20 for accommodating the DNA chains to be detected, one ion-sensitive film 3 is arranged to correspond to each of the openings 20, and the ion-sensitive films 3 do not contact with one another to avoid test disorder.

Furthermore, FIG. 1 only shows a possible manner for arranging the openings 20 in the openings defining layer 2. In some embodiments of the present disclosure, the openings 20 can be uniformly arranged right above or obliquely above each of the display units 11 in the display panel 1 (i.e., as shown in FIG. 1). Alternatively, each of the openings 20 can also be concentrated in a peripheral region of the display panel 1, provided that the arrangement order of the openings 20 corresponding with the transistor 12 in each of the display units 11 is clearly labelled, thus facilitating the gene sequencing.

In some embodiments of the present disclosure, in view of the fabricating process difficulty of the chip and the gene test accuracy, the openings 20 can be micropores with an aperture (diameter) of 1-100 μm, thus facilitating preparing and placing DNA microbeads.

In the gene test chip according to embodiments of the present disclosure, during gene sequencing, the nucleotide molecules continuously flow one by one over the openings 20 on the chip. In case a deoxy-ribo nucleoside triphosphate is complementarily paired with the DNA molecule in one of the openings 20, a hydrogen ion will be released and induce a Nernst potential on the surface of the ion-sensitive film 3. The potential signal is transmitted to the gate 12g, so as to turn on the transistor 12 corresponding with that opening 20. A hydrogen ion is not released in the opening 20 where the DNA molecule is not complementarily paired, and no Nernst potential is induced on the surface of the ion-sensitive film 3. Accordingly, the transistor 12 corresponding with that opening 20 is not turned on, and the change in display of the display panel 1 is not caused. By means of a corresponding processor, the display change can be converted into corresponding digital electronic information, so as to obtain a base type in the DNA chains under test, and thus conduct gene sequencing. The gene sequencing chip adopts the principle of the ionic semiconductor sequencing technique, and there is no need for fluorescence labeling deoxy-ribo nucleoside triphosphate, or for a laser source and an optical system. Thus, the gene sequencing chip is simpler in structure, contains less transistors, can be fabricated with reduced difficulty, and efficiently reduces the sequencing time and cost.

On the basis of the foregoing, embodiments of the present disclosure further provide a gene sequencing device, including the gene sequencing chip as described above and a processor, where the processor is configured to obtain the base sequence of DNA chains according to the display change which is produced on the display panel 1 during gene sequencing.

As is known from the above description about the testing principle, in particular, the complementary pairing occurs between the DNA microbeads which include DNA chains and are added into the openings 20 during gene sequencing and one of the deoxy-ribo nucleoside triphosphates, the electrical signal is produced on the ion-sensitive film 3 to turn on the transistor 12, and the base sequence of DNA chains is obtained from the display change on the display panel 1.

Moreover, embodiments of the present disclosure further provide a gene sequencing method by means of the gene sequencing chip as described above. By taking four kinds of deoxy-ribo nucleoside triphosphates in the common sequencing as an example, the sequencing method includes:

adding DNA microbeads which include DNA chains into the openings 20 for PCR amplification;

adding four kinds of deoxy-ribo nucleoside triphosphates into the openings successively 20, where after complementary pairing the DNA chains and one of the four kinds of deoxy-ribo nucleoside triphosphates occurs, an electrical signal is produced on the ion-sensitive film 3 to turn on the transistor 12, so that a display change is produced on the display panel 1;

obtaining the base sequence of DNA chains according to the produced display change.

In case the display panel 1 is a display panel which operates on the electrowetting principle, the structure will be simpler, and the change in display will be more obvious and recognizable. Thus in some embodiments of the present disclosure, the display panel 1 adopts a display panel operating on the electrowetting principle. The specific structure and test process of the display panel 1 will be described in detail hereinafter with reference to the following embodiments.

In some embodiments, as shown in FIG. 2, the display panel 1 includes a first substrate 10 and a second substrate 18 which are arranged oppositely; and a dielectric layer 14, a first fluid layer 15 and an electrically conductive second fluid layer 16 which are arranged in a space between the first substrate 10 and the second substrate 18. The first fluid layer 15 is arranged on a side of the second fluid layer 16 close to the electrode 13. The first fluid layer 15 and the second fluid layer 16 have different colors. In case no electric field is formed between the electrode 13 and the second fluid layer 16, the first fluid layer 15 is spread on a surface of the dielectric layer 14. As shown in FIG. 3, in case an electric field is formed between the electrode 13 and the second fluid layer 16, the first fluid layer is split into a plurality of subportions 150. The subportions 150 are concentrated in regions of the dielectric layer 14 where the corresponding transistors 12 are located, and do not contact with one another. The transistor 12 and the electrode 13 are arranged on the first substrate 10. The gene sequencing chip further includes a protection layer 17 which covers the transistor 12 and the electrode 13. The ion-sensitive film 3 is connected with the gate 12g through a via hole 170 in the protection layer 17.

The testing principle is described hereinafter.

During sequencing, the nucleotide molecules continuously flow one by one over the openings 20 on the chip. In case a complementary pairing between a deoxy-ribo nucleoside triphosphate and the DNA molecule occurs in the openings 20, a hydrogen ion will be released and induce a Nernst potential on the surface of the ion-sensitive film 3. The potential signal is transmitted to the gate 12g, so as to turn on the transistor 12 corresponding with the opening 20. After a corresponding electrical signal is applied to the source 12s, the electrode 13 is charged through the drain 12d, and a certain potential (e.g., which can connect the liquid of the second fluid layer 16 with a ground potential) is applied to the electrically conductive second fluid layer 16. When the energy of the electric field is larger than the surface energy of liquid of the first fluid layer 15, on basis of the electrowetting principle, the first fluid layer 15 which was able to spread on (i.e., wetting) the dielectric layer 14 begins splitting, and droplets are produced. Namely, the first fluid layer 15 becomes difficult to be spread on the surface of the dielectric layer 14 due to the action of the electric field. Since the electrode 13 is absent at a bottom of the transistor 12, the first fluid layer 15 is split into the subportions 150 which are concentrated in regions of the dielectric layer 14 where the corresponding transistors 12 are located, and which do not contact with one another. In this case, the bottom of the micropore becomes transparent. Since the first fluid layer 15 and the second fluid layer 16 have different colors, a pattern of the second fluid layer 16, which are spaced apart by the plurality of subportions 150 that do not contact with one another, will be displayed at a side of the display panel 1 away from the bottom of the openings 20. The pattern displayed at the bottom of the display panel 1 is captured by corresponding imaging circuits, and the chemical information is converted into light information for gene sequencing.

In some embodiments, as shown in FIG. 2, the dielectric layer 14 can be arranged on a side of the first fluid layer 15 away from the second fluid layer 16. In particular, during fabrication, the dielectric layer 14 can be deposited on the bottom surface of the first substrate 10, and then the first fluid layer 15 is encapsulated to further reduce fabricating process difficulty.

In some embodiments, the dielectric layer 14 for example can be a hydrophobic layer, and the hydrophobic layer can be made from a liquid including a fluoropolymer (e.g. polytetrafluoroethylene). The first fluid layer 15 is an oil film, the oil film can be made from a liquid which includes at least one of hexadecane and silicone, and in which at least one of a pigment and a dye is dissolved. In case the electric field is absent, the first fluid layer 15 can be wetted and spread on the hydrophobic layer, since the first fluid layer 15 has a same hydrophilcity or hydrophobicity as the hydrophobic layer. The electrically conductive second fluid layer 16 can be made from a liquid including water or a salt solution.

In order to increase the color contrast effect between the first fluid layer 15 and the second fluid layer 16 during a gene test, and to increase the test accuracy, in some embodiments of the present disclosure, the first fluid layer 15 is black in color, i.e., a black pigment and/or a black dye is dissolved in a solvent of at least one of hexadecane and silicone. In contrast, the second fluid layer 16 is a color other than black (and can also be transparent).

Here, the dye refers to an organic compound which can dye a matrix (i.e., the solvent of at least one of hexadecane and silicone in the embodiment described with reference to FIG. 2) into a certain color (e.g. black). The pigment refers to an organic or inorganic colored compound which is colored and insoluble in a medium (i.e., the solvent of at least one of hexadecane and silicone), and primarily is granular, so that a corresponding color (e.g. black) is formed due to refraction by the pigment dispersed in the medium.

It is noted that, the term “layer” in the first fluid layer 15 and the second fluid layer 16 does not limit the geometrical shape of the fluid, and “layer” is not limited to a description of the spread state. Due to flowability of liquid of the first fluid layer 15, on basis of the electrowetting principle, the spreading state of the first fluid layer 15 on the dielectric layer 14 will change accordingly under the action of the electric field.

Embodiments of the present disclosure further provide a gene sequencing device, which includes the gene sequencing chip in the embodiment described with FIG. 2; an imaging circuit, which is configured to record a pattern displayed at the bottom of the display panel 1 away from the openings 20; and a processor, which is configured to obtain the base sequence of DNA chains according to the displayed pattern.

Here, according to the above testing principle, the imaging circuit is configured to record the pattern which is displayed at the bottom of the display panel 1 away from the openings 20, when the first fluid layer 15 is split into the plurality of subportions 150 which are concentrated in regions of the dielectric layer 14 where the corresponding transistors 12 are located and do not contact with one another.

The imaging circuit for example can include an imaging device which is a charge coupled device (CCD) or a complementary metal oxide semiconductor (CMOS).

The processor can be connected with the imaging circuit so as to obtain the displayed pattern which is recorded by the imaging circuit. The processor can be a logic-arithmetic device with a data handling capacity and/or program execution capability, such as a central processing unit (CPU), a field programmable gate array (FPGA), a Microcontroller Unit (MCU), or a digital signal processor (DSP).

A connection can be through a wireless network, a wired network, and/or any combination of the wireless network and the wired network. The network can include a local area network, an Internet, a telecommunication network, an internet of things based on the internet and/or the telecommunication network, and/or any combination of the above networks.

Embodiments of the present disclosure further provide a gene sequencing method on basis of the gene sequencing chip in the above embodiments. The sequencing method includes the following steps.

In step S01, DNA microbeads which include DNA chains are added into the openings 20 for PCR amplification.

In step S02, four kinds of deoxy-ribo nucleoside triphosphates (dNTPs) are added into the openings successively 20, and a selected potential (e.g., a ground potential, i.e., a zero potential) is applied to the second fluid layer 16, so that under the action of the electric field between the second fluid layer 16 and the electrode 13 during complementary pairing in the openings 20, the first fluid layer 15 is split into subportions 150 which are concentrated in regions of the dielectric layer 14 where the corresponding transistors 12 are located and which do not contact with one another.

In step S03, when the first fluid layer 15 is split into the plurality of subportions 150 which do not contact with one another, the pattern displayed at the bottom of the display panel 1 away from the openings 20 is obtained.

In step S04, the base type on the DNA chains is determined according to the specific type of the deoxy-ribo nucleoside triphosphates which are added when the pattern is produced.

In particular, when the transistor 12 corresponding with a micropore is turned on, in case the deoxy-ribo nucleoside triphosphate added to the micropore is triphosphate adenine deoxy-ribo nucleotide, the base on the DNA chains to be measured is thymine; in case the deoxy-ribo nucleoside triphosphate added to the micropore is triphosphate thymine deoxy-ribo nucleotide, the base on the DNA chains to be measured is adenine; in case the deoxy-ribo nucleoside triphosphate added to the micropore is triphosphate cytosine deoxy-ribo nucleotide, the base on the DNA chains to be measured is guanine; and in case the deoxy-ribo nucleoside triphosphate added to the micropore is triphosphate guanine deoxy-ribo nucleotide, the base on the DNA chains to be measured is cytosine.

On the basis of the foregoing, in case the four kinds of deoxy-ribo nucleoside triphosphates added to the micropore are four reversibly terminating deoxy-ribo nucleoside triphosphates, the gene sequencing method further includes the following steps:

removing the four reversibly terminating deoxy-ribo nucleoside triphosphates which are successively added into the openings 20 by washing, and adding a sulfhydryl reagent for detecting the base type at a subsequent position of the DNA chains.

Namely, once the detection of the base type at a position of the DNA is complete, it is required to remove the reversibly terminating deoxy-ribo nucleoside triphosphate added to the micropore by washing, and to add the sulfhydryl reagent. In contrast with the common deoxy-ribo nucleoside triphosphate, a terminal position of 3′ hydroxyl in the reversibly terminating deoxy-ribo nucleoside triphosphate is connected with an azide group (which has a property of being cut chemically), and a phosphodiester bond can not be formed during a DNA synthesis process. Namely, a single base is allowed to be incorporated during each cycle, and thus the synthesis of DNA is interrupted. After the type of nucleotide which is polymerized to each template sequence during the first round of reaction is obtained, the sulfhydryl reagent is added to chemically cut these groups. As a result, the azide group is broken, so as to recover the stickiness of the terminal of 3′ hydroxyl. Namely, a hydroxyl is formed at the original position, so that a second nucleotide can be polymerize at this position for detecting the base type at the subsequent position. The detection method is identical with the above method, and is not repeated for simplicity. This is repeated, until each of the template sequences is polymerized into a double strand. The sequence of each template DNA fragment can be obtained from the statistical data about light information of display pattern which is gathered in each round.

In the gene test chip according to embodiments of the present disclosure, during gene sequencing, the nucleotide molecules continuously flow one by one over the openings on the chip. In case a deoxy-ribo nucleoside triphosphate is complementarily paired with the DNA molecule in the openings, a hydrogen ion will be released and induce a Nernst potential on the surface of the ion-sensitive film. The potential signal is transmitted to the gate, so as to turn on the transistor corresponding with the opening. A hydrogen ion is not released in the openings where the DNA molecule is not complementarily paired, and no Nernst potential is induced on the surface of the ion-sensitive film. Accordingly, the transistor corresponding with the opening is not turned on, and the change in display of the display panel is not caused. By means of a corresponding processor, the display change can be converted into corresponding digital electronic information, so as to obtain a base type in the DNA chains under test, and thus conduct gene sequencing. The gene sequencing chip adopts the principle of the ionic semiconductor sequencing technique, and there is no need for fluorescence labeling deoxy-ribo nucleoside triphosphate, or for a laser source and an optical system. Thus, the gene sequencing chip is simpler in structure, contains less transistors, can be fabricated with reduced difficulty, and efficiently reduces the sequencing time and cost.

A person with ordinary skill in the art can make various modifications and variations to the present disclosure without departing from the spirit and the scope of the present disclosure. In this way, provided that these modifications and variations of the present disclosure belong to the scopes of the claims of the present disclosure and the equivalent technologies thereof, the present disclosure also intends to encompass these modifications and variations.

1. A gene sequencing chip, comprising:
a display panel which comprises a plurality of display units, wherein each of the display units comprises a transistor and an electrode connected with a first pole of the transistor;
an opening defining layer which is arranged on the display panel and comprises openings corresponding with the display units in a one-to-one manner; and
an ion-sensitive film which is at least partially arranged in the openings and is connected with a control electrode of the transistor.

2. The gene sequencing chip of claim 1, wherein the display panel further comprises:
a first substrate and a second substrate which are arranged oppositely; and
a dielectric layer, a first fluid layer, and an electrically conductive second fluid layer which are arranged in a space between the first substrate and the second substrate.

3. The gene sequencing chip of claim 2, wherein the first fluid layer is arranged on a side of the second fluid layer close to the electrode,
wherein the first fluid layer has a color different from the second fluid layer, and
the electrode and the second fluid layer are configured in such a manner that in case no electric field is formed, the first fluid layer is spread on a surface of the dielectric layer; and in case an electric field is formed, the first fluid layer is split into a plurality of subportions which are concentrated in regions of the dielectric layer where the corresponding transistors are located and which do not contact with one another.

4. The gene sequencing chip of claim 3, wherein the transistor and the electrode are arranged on the first substrate.

5. The gene sequencing chip of claim 1, further comprising a protection layer which covers the transistor and the electrode, wherein the ion-sensitive film is connected with the control electrode through a via hole in the protection layer.

6. The gene sequencing chip of claim 1, wherein the openings are micropores with an aperture of 1-100 μm.

7. The gene sequencing chip of claim 3, wherein the dielectric layer is arranged on a side of the first fluid layer away from the second fluid layer.

8. The gene sequencing chip of claim 3, wherein the dielectric layer is a hydrophobic layer, and the first fluid layer is an oil film.

9. The gene sequencing chip of claim 8, wherein the hydrophobic layer is made from a liquid comprising a fluoropolymer.

10. The gene sequencing chip of claim 8, wherein the oil film is made from a liquid which comprises at least one of hexadecane and silicone, and in which at least one of a pigment and a dye is dissolved.

11. The gene sequencing chip of claim 2, wherein the first fluid layer has a black color.

12. The gene sequencing chip of claim 1, wherein the ion-sensitive film is made from Si₃N₄.

13. The gene sequencing chip of claim 1, further comprising a peripheral circuitry, wherein a second pole of the transistor is electrically connected with the peripheral circuitry through a signal line.

14. A gene sequencing device, comprising:
the gene sequencing chip of claim 1; and
a processor which is configured to obtain the base sequence of DNA chains according to a display change produced on the display panel during gene sequencing.

15. The gene sequencing device of claim 14, further comprising: an imaging circuit which is configured to record a pattern displayed at a bottom of the display panel away from the openings, wherein the processor is configured to obtain the base sequence of DNA chains according to the pattern.

16. A gene sequencing method by using the gene sequencing chip of claim 1, comprising:
adding DNA microbeads which comprise a plurality of DNA chains into the openings for PCR amplification;
adding a plurality of kinds of deoxy-ribo nucleoside triphosphates into the openings successively, where the plurality of DNA chains are complementarily paired with one of the plurality of kinds of deoxy-ribo nucleoside triphosphates to produce an electrical signal on the ion-sensitive film which turns on the transistor, so that a display change is produced on the display panel; and
obtaining the base sequence of DNA chains according to the display change.

17. The gene sequencing method of claim 16, wherein the display panel further comprises:
a first substrate and a second substrate which are arranged oppositely; and
a dielectric layer, a first fluid layer, and an electrically conductive second fluid layer which are arranged in a space between the first substrate and the second substrate, and
wherein the step of adding the plurality of kinds of deoxy-ribo nucleoside triphosphates into the openings successively, wherein the DNA chains are complementarily paired with one of the plurality of kinds of deoxy-ribo nucleoside triphosphates to produce an electrical signal on the ion-sensitive film which turns on the transistor, so that the display change is produced on the display panel comprises:
adding the plurality of kinds of deoxy-ribo nucleoside triphosphates into the openings successively, and
applying a selected potential to the second fluid layer, so that under the action of the electric field between the second fluid layer and the electrode during complementary pairing in the openings, the first fluid layer is split into subportions which are concentrated in regions of the dielectric layer where the corresponding transistors are located and which do not contact with one another.

18. The gene sequencing method of claim 17, further comprising:
obtaining a pattern which is displayed at a bottom of the display panel away from the openings after the first fluid layer is split into the subportions which do not contact with one another.

19. The gene sequencing method of claim 18, wherein obtaining the base sequence of DNA chains according to the display change comprises:
determining the base type on the DNA chains according to the specific type of the deoxy-ribo nucleoside triphosphates which are added when the pattern is produced.

20. The gene sequencing method of claim 17, wherein the plurality of kinds of deoxy-ribo nucleoside triphosphates comprise a plurality of kinds of reversibly terminating deoxy-ribo nucleoside triphosphates, and wherein the gene sequencing method further comprises:
removing the plurality of kinds of reversibly terminating deoxy-ribo nucleoside triphosphates which are added into the openings successively by washing, and
adding a sulfhydryl reagent for detecting the base type at a subsequent position of the DNA chains.
